SEQUENCE LISTING (9 sequences)

SEQ ID NO: 1 - protein, 9 aa, HIV
RKKRRQRRR

SEQ ID NO: 2 - DNA, 762 bp, Homo sapiens
  CDS (1)..(762)
atggcgaaccttggctgctggatgctggttctctttgtggccacatggagtgacctgggcctctgcaagaagcgcccgaagcctggaggatggaacactgggggcagccgatacccggggcagggcagccctggaggcaaccgctacccacctcagggcggtggtggctgggggcagcctcatggtggtggctgggggcagcctcatggtggtggctgggggcagccccatggtggtggctggggacagcctcatggtggtggctggggtcaaggaggtggcacccacagtcagtggaacaagccgagtaagccaaaaaccaacatgaagcacatggctggtgctgcagcagctggggcagtggtggggggccttggcggctacatgctgggaagtgccatgagcaggcccatcatacatttcggcagtgactatgaggaccgttactatcgtgaaaacatgcaccgttaccccaaccaagtgtactacaggcccatggatgagtacagcaaccagaacaactttgtgcacgactgcgtcaatatcacaatcaagcagcacacggtcaccacaaccaccaagggggagaacttcaccgagaccgacgttaagatgatggagcgcgtggttgagcagatgtgtatcacccagtacgagagggaatctcaggcctattaccagagaggatcgagcatggtcctcttctcctctccacctgtgatcctcctgatctctttcctcatcttcctgatagtgggatga

SEQ ID NO: 3 - protein, 253 aa, Homo sapiens
MANLGCWMLVLFVATWSDLGLCKKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGGYMLGSAMSRPIIHFGSDYEDRYYRENMHRYPNQVYYRPMDEYSNQNNFVHDCVNITIKQHTVTTTTKGENFTETDVKMMERVVEQMCITQYERESQAYYQRGSSMVLFSSPPVILLISFLIFLIVG

SEQ ID NO: 4 - protein, 7 aa, HIV
KKRPKPG

SEQ ID NO: 5 - protein, 86 aa, Human immunodeficiency virus
MEPVDPRLEPWKHPGSQPKTACTNCYCKKCCFHCQVCFITKALGISYGRKKRRQRRRAHQNSQTHQASLSKQPTSQPRGDPTGPKE

SEQ ID NO: 6 - protein, 10 aa, HIV
KPSKPKTLNK

SEQ ID NO: 7 - protein, 5 aa, HIV
  MISC_FEATURE (2)..(2): Xaa is Arginine or Lysine
  MISC_FEATURE (4)..(4): Xaa can be any amino acid
  MISC_FEATURE (5)..(5): Xaa is Arginine or Lysine
KXRXX

SEQ ID NO: 8 - DNA, 765 bp, Mus musculus
  CDS (1)..(765)
atggcgaaccttggctactggctgctggccctctttgtgactatgtggactgatgtcggcctctgcaaaaagcggccaaagcctggagggtggaacaccggtggaagccggtatcccgggcagggaagccctggaggcaaccgttacccacctcagggtggcacctgggggcagccccacggtggtggctggggacaaccccatgggggcagctggggacaacctcatggtggtagttggggtcagccccatggcggtggatggggccaaggagggggtacccataatcagtggaacaagcccagcaaaccaaaaaccaacctcaagcatgtggcaggggctgcggcagctggggcagtagtggggggccttggtggctacatgctggggagcgccatgagcaggcccatgatccattttggcaacgactgggaggaccgctactaccgtgaaaacatgtaccgctaccctaaccaagtgtactacaggccagtggatcagtacagcaaccagaacaacttcgtgcacgactgcgtcaatatcaccatcaagcagcacacggtcaccaccaccaccaagggggagaacttcaccgagaccgatgtgaagatgatggagcgcgtggtggagcagatgtgcgtcacccagtaccagaaggagtcccaggcctattacgacgggagaagatccagcagcaccgtgcttttctcctcccctcctgtcatcctcctcatctccttcctcatcttcctgatcgtgggatga

SEQ ID NO: 9 - protein, 254 aa, Mus musculus
MANLGYWLLALFVTMWTDVGLCKKRPKPGGWNTGGSRYPGQGSPGGNRYPPQGGTWGQPHGGGWGQPHGGSWGQPHGGSWGQPHGGGWGQGGGTHNQWNKPSKPKTNLKHVAGAAAAGAVVGGLGGYMLGSAMSRPMIHFGNDWEDRYYRENMYRYPNQVYYRPVDQYSNQNNFVHDCVNITIKQHTVTTTTKGENFTETDVKMMERVVEQMCVTQYQKESQAYYDGRRSSSTVLFSSPPVILLISFLIFLIVG

(19) United States
(12) Patent Application Publication
(10) Pub. No.: US 2008/0027025 A1
(43) Pub. Date: Jan. 31, 2008

(54) Protection From And Treatment Of Prion Protein Infection

(75) Inventors: Steven F. Dowdy, La Jolla, CA (US); Jehangir S. Wadia, La Jolla, CA (US)

Correspondence Address: BUCHANAN, INGERSOLL & ROONEY LLP, P.O. BOX 1404, ALEXANDRIA, VA, 22313-1404, US

(73) Assignee: The Regents of the University of California, Oakland, CA (US)

(21) Appl. No.: 11/661,239

(22) PCT Filed: Aug. 26, 2005
(86) PCT No.: PCT/US05/30322

§ 371 Date: Aug. 1, 2007

Related U.S. Application Data
(60) Provisional application No. 60/605,043, filed on Aug. 27, 2004.

(51) Int. Cl. A61K 31/4965; A01N 43/04; A23B 4/20; A61K 31/10; A61K 31/216; A61P 31/00; C12N 5/00; A61K 31/715; A61K 31/335; A61K 31/352; A61K 31/4164

(52) U.S. Cl. 514/56; 426/332; 435/375; 514/255.06; 514/396; 514/450; 514/453; 514/529; 514/54; 514/709; 514/711; 514/789

(57) ABSTRACT

The disclosure for the first time provides an understanding of the mechanism of prion protein infection: prion proteins contain a cationic protein transduction domain (PTD) that interacts with the cell surface such that it induces macropinocytosis and enters the cytoplasm.

### CROSS-REFERENCE TO RELATED APPLICATIONS

This application claims priority under 35 U.S.C. §119 to U.S. Provisional Application Ser. No. 60/605,043, filed Aug. 27, 2004, the disclosure of which is incorporated herein by reference.

### STATEMENT REGARDING FEDERALLY SPONSORED RESEARCH

The invention was funded in part by Grant No. CA96098 awarded by the National Institutes of Health. The government may have certain rights in the invention.

### TECHNICAL FIELD

This invention relates to methods and compositions useful for inhibiting prion infections and more particularly to methods for inhibiting the production Prp^{Sc}.

### BACKGROUND

Infectious agents such as bacteria, fungi, parasites, and viroids have well established methods of infection and methods of control that involve various forms of antibiotics, antivirals, and the like.

A family of pathogenic agents has appeared and has been reported in scientific publications. These have been referred to as “prions” and present one of the greatest challenges facing the health care industry today. Prions are infectious particles that differ from bacteria and other known infectious agents. While there is no firm evidence on the exact structure of prions, a number of diseases have been identified recently both in humans and animals, that appear to be attributable to prions. Human diseases attributed to prions include Kuru, Creutzfeldt-Jakob disease (CJD), Gerstmann-Straussler-Scheinker disease (GSS), and Fatal Familial Insomnia (FFI).

In addition to prion diseases of humans, disorders of animals are included in the group of known prion diseases. Scrapie of sheep and goats is perhaps the most studied animal prion disease. Several lines of inquiry have suggested a link between variant CJD and a preceding epidemic of bovine spongiform encephalopathy (BSE). No successful therapeutic treatments have been developed and as a result these diseases are typically fatal.

Groups possibly at risk of infection include subjects who may come into contact with infected medical instruments during surgery, medical staff dissecting infected materials, and healthcare workers responsible for cleaning and sterilizing instruments. There are also concerns that groups at risk may be broadened to include veterinarians, abattoir workers, butchers in contact with bovine or beef primarily in Europe and more recently persons receiving blood transfusions or organs from donors incubating a prion disease.

### SUMMARY

The N-terminus of the prion protein contains a cationic protein transduction domain (PTD) that interacts with the cell surface, inducing macropinocytosis and promotes escape from the macropinosome vesicle into the cytoplasm. Treatment of cells being exposed to prion proteins with an anionic agent, for example, heparin, an anionic polymer, neutralizes the cationic charge in the prion's PTD and prevents entrance or infection of cells.

Furthermore the invention provides methods and compositions useful to inhibit uptake of prion proteins that thus inhibit prion-associated diseases and disorders by inhibiting macropinocytosis. As described further herein, prion proteins are taken into cells by a process called macropinocytosis. Thus, inhibitors of macropinocytosis are useful in inhibiting cellular uptake of prions and inhibition of prion-associated diseases and disorders.

Accordingly, it is possible to reduce prion infectivity with concomitant oral treatment with anionic compositions and/or macropinocytosis inhibitors during consumption of contaminated food stuff. The anionic agent serves to neutralize the prion's PTD and prevents its escape from the intestinal track into the blood stream and eventually the CNS. Likewise, treatment of infected subjects with anionic agents (e.g., heparin), as well as other anionic polymers, will neutralize and prevent further spread of the disease. Furthermore, the use of macropinocytosis inhibitors alone or in combination with anionic agents can prevent the process by which infectious prions enter a cell.

Accordingly, the invention provides methods and compositions useful for prevention of prion protein infection from, for example, consumption of beef contaminated by Mad Cow Disease of which prion protein is a causative agent.

The invention provides methods of inhibiting infection by an infectious prion, comprising contacting a cell susceptible to infection with a prion with an inhibiting effective amount of an agent selected from the group consisting of an anionic agent, a glycosaminoglycan, an agent that sequesters cholesterol (e.g., nystatin), a macropinocytosis inhibitor and any combination thereof, prior to, concomitant with, and/or following contact of the cell with the prion, for a sufficient time and under sufficient conditions such that the anionic agent inhibits uptake of the prion. In one aspect, the contacting is in vivo. In another aspect the cell or subject being contacted is a human, bovine, sheep, mink, or other organism susceptible to prion infection and/or prion associated diseases and disorders.

The invention also provides a method of inhibiting the infectivity of a prion comprising contacting a sample suspected of containing a prion with an agent selected from the group consisting of an anionic agent, a glycosaminoglycan, an agent that sequesters cholesterol, a macropinocytosis inhibitor and any combination thereof.

The invention provides a method of treating a subject having or at risk of becoming infected with an infectious prion, comprising administering to the subject an agent selected from the group consisting of an anionic agent, a glycosaminoglycan, an agent that sequesters cholesterol, a macropinocytosis inhibitor and any combination thereof in an amount sufficient to inhibit prion infectivity or spread.

The invention further provides a method of inhibiting the production of a pathological prion protein comprising contacting a cell susceptible to infection with a prion or infected with a pathological prion with an inhibiting effective amount of an agent selected from the group consisting of an anionic agent, a glycosaminoglycan, an agent that sequesters cholesterol, a macropinocytosis inhibitor and any combination thereof, prior to, concomitant with, and/or following contact of the cell with the prion, for a sufficient time and under sufficient conditions such that the anionic agent inhibits uptake of the prion.

The invention also provides a composition for use in inhibiting prion infectivity in a subject comprising an agent selected from the group consisting of an anionic agent, a glycosaminoglycan, an agent that sequesters cholesterol, a macropinocytosis inhibitor and any combination thereof in unit dose form.

In another aspect of the invention, peptides useful for delivery of molecules of interest are provided. The peptide comprises a sequence of between 7 and 10 amino acids, wherein at least 4 of the amino acids are basic amino acids such as lysine, arginine and histidine. In one embodiment, the PTD domain consists of from about 7 to 10 amino acids, wherein at least 4 amino acids are lysine or arginine. In another aspect, the PTD domain consists of a sequence of about 7 to 10 amino acids and contains the sequence KX₁RX₂X₁, wherein X₁ is R or K and X₂ is any amino acid (SEQ ID NO:7).

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A-C shows exogenous rPrP^{C} co-localizes with TAT-fusion proteins in cells. (a) Alignment of putative rPrP^{C} transduction domain (SEQ ID NO:4) with the HIV-1 TAT PTD (SEQ ID NO:1) and schematic of rPrP^{C}-Cre recombinase fusion proteins. Also shown is an internal cationic domain (SEQ ID NO:9 from amino acid 100-109). (b) Co-localization of rPrP^{C} (residues 23-231) and TAT-Cre. N2a cells were treated with rPrP^{C}-Alexa546 and TAT-Cre-Alexa488, and assayed by live cell confocal microscopy. The “overlay” indicates areas of co-localization. Scale bar=5 μm. (c) N2a cells preincubated for 30 min with 50 μg/mL heparin or 5 mM nystatin followed by rPrP^{C}Alexa546 protein incubation for 2 h prevented surface binding and internalization, respectively. Scale bar=10 μm.

FIG. 2A-C shows cellular uptake of rPrP^{C} occurs by endocytosis. (a) Reporter cells containing loxP-STOP-loxP GFP gene were treated with indicated proteins, incubated overnight and assayed for GFP-positive cells by flow cytometry (±SD). (b) Reporter cells were treated with rPrP^{C} (23-90)-Cre in the presence of heparin or chondroitin sulfate B and assayed for GFP-positive cells by flow cytometry (±SD). (c) N2a cells were transfected with Cav1α-GFP expression plasmid, followed by treatment with rPrP^{C}-546 (red) and live cell confocal microscopy. Scale bar=5 μm. (d) N2a cells co-transfected with pDyn^{K44A}-HA dominant-negative and pZ/EG loxP-stop-loxP GFP reporter plasmids were treated with rPrP^{C}(23-90)-Cre protein. Scale bar=25 μm. (e) N2a cells co-transfected with pDyn^{K44A}-HA and pEGFP (constitutive GFP expression) plasmids (10:1) were incubated with fluorescent transferrin-TMR. Arrows indicate Dyn^{K44A}/EGFP transfected cells. Scale bar=20 μm.

FIG. 3A-B shows Cellular uptake of rPrP protein occurs by macropinocytosis. (a) N2a cells were pretreated with either 100 μM EIPA or 5 mM cytochalasin D for 30 min prior the addition of fluorescently labeled recombinant rPrP^{C}-Alexa546 for 2 h followed by washing, trypsinization confocal microscopy. Scale bar=10 μm. (b) N2a cells were treated with 0.5 mg/mL 70 kDa dextran-FITC for, 30 min. in the presence of increasing concentrations of rPrP^{C} (0, 0.25, 0.5, 1.0 or 2.0 μM). Fluid phase uptake of dextran was measured by flow cytometry (values±SD, Student's T-test: **=p<0.02, ***=p<0.002).

FIG. 4A-B shows pathological PrP^{Sc} conversion of cellular PrP^{C} requires macropinocytosis. N2aPK1 cells were exposed to 10-5 dilution of RML PrPSc-infected brain homogenate and increasing concentrations of the macropinocytosis inhibitor EIPA (25, 50 and 100 μM) for 48 h. Infected cells were passaged three times (1:10), then grown on cover slips, blotted, digested with proteinase K (PK) and probed with anti-PrP antibodies (a) and Ponceau S staining (b).

### DETAILED DESCRIPTION

The structure of prions has been the subject of intense investigation and different points of view have been expressed. Some scientists believe they are extremely small viruses, while most experts now believe that prions are actually infectious proteins without a DNA or RNA core. More particularly the consensus now is that the PrP gene of mammals expresses a protein which can be the soluble, non-disease, cellular form PrP^{c} or can be an insoluble disease form PrP^{sc}. Many lines of evidence indicate that prion diseases result from the transformation of the normal cellular form into the abnormal PrP^{Sc} form. There is no detectable difference in the amino acid sequence of the two forms. The PrP^{c} form is composed of a membrane associated 33-35 kDa protein which degrades on digestion with protease K. However the PrP^{Sc} form has an altered conformational form, in particular having a high level of β-sheet conformation. Properties of PrP^{Sc} useful in diagnosing the infective altered conformational form are a protease resistant core of 27-30 kDa. Another distinctive feature of the altered conformational infective form is that it acquires a hydrophobic core.

Transmissible spongiform encephalopathies, including variant Creutzfeldt-Jakob disease in humans and mad cow disease in cattle are fatal neurodegenerative disorders that are characterized by template-directed protein misfolding of the host wild-type cellular prion protein (PrP^{C}). PrP^{C} is a cell surface-associated protein whose normal physiologic function is unclear; however, host cell exposure to infectious, protease-resistant prion protein (PrP^{Sc}) results in the conformational conversion of endogenous PrP^{C} to the pathological PrP^{Sc} isoform. The presence of small amounts of cytoplasmic PrP has been shown to induce neuropathologies. Until now, the mechanism that exogenous PrP uses to enter cells remained unknown.

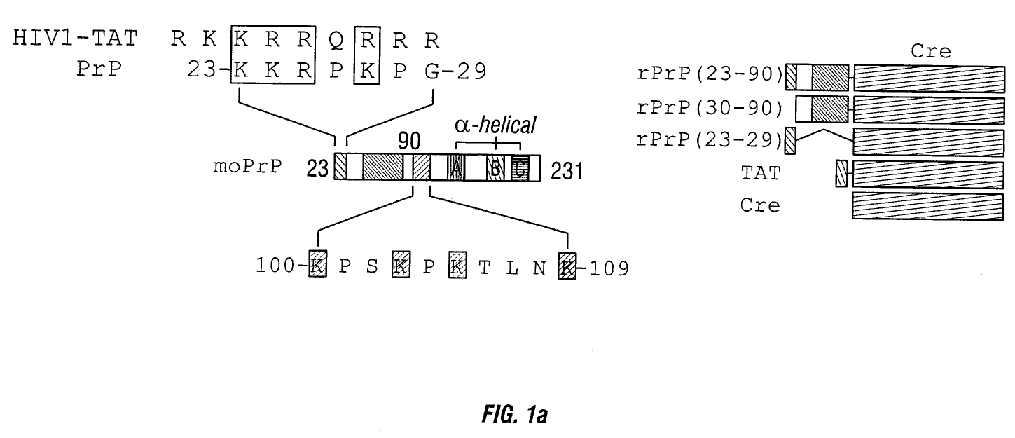

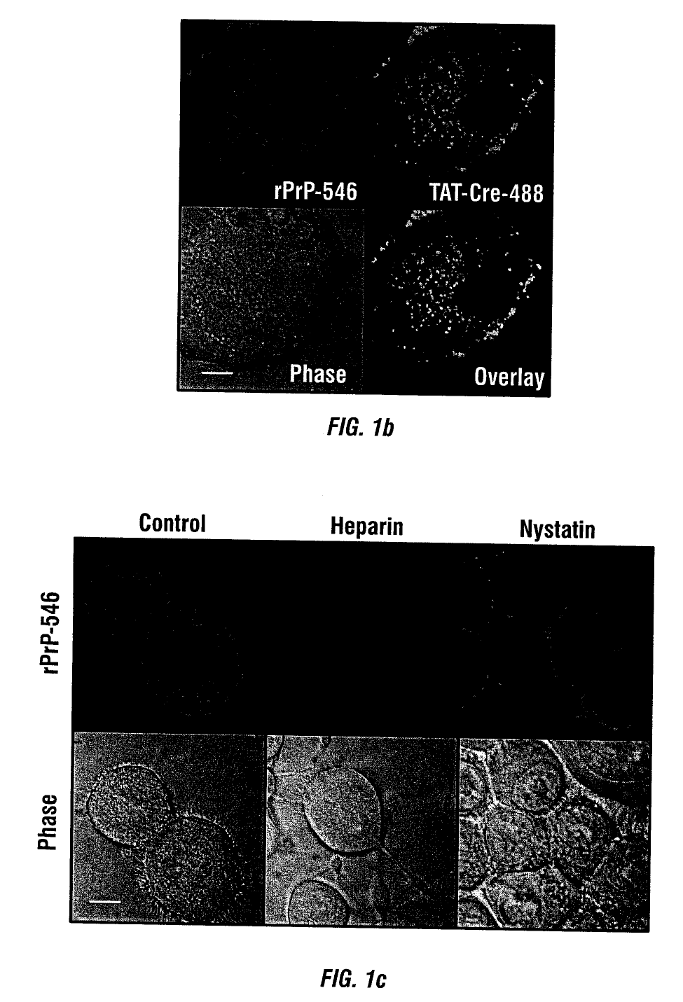

The invention is based, in part, upon the identification of a mechanism of prion infection through macropinocytosis. The invention also provides a map of a cationic protein transduction domain (PTD) at the N-terminus (residues 23-29) of prion proteins (see, e.g., FIG. 1a).

Prion proteins exist in at least two states in nature. The first state is what is referred to as the cellular state or PrP^{C}. PrP^{C} is susceptible to protease digestion. The second state is the mutant state in which a conformation change in the prion proteins structure has occurred leading to protease resistance causing spongiform encephalopathies. This disease form is referred to as PrP^{Sc}. The polynucleotide sequence encoding a prion protein and polypeptide sequence of a prion protein are provided in SEQ ID NOs:2 and 3, respectively.

Prion proteins have a number of domains associated with their structure. For example, prion proteins have a signal domain (e.g., amino acids 1-22 of SEQ ID NO:3) and a protein transduction domain (PTD) (e.g., amino acids 23-29 of SEQ ID NO:3). A further cationic domain that plays a role in cellular uptake and/or release of prion proteins can be found at amino acid 101-110 of SEQ ID NO:3. The mature protein is about 33-35 kDA and comprises a sequence from amino acid 23-230 of SEQ ID NO:3. One of skill in the art will be able to identify homologous domain in prion proteins from another of other species using alignment tools known and used in the art.

The invention further provides a PTD peptide useful to cause uptake of heterologous molecules comprising a sequence of between 7 and 10 amino acids, wherein at least 4 of the amino acids are basic amino acids such as lysine, arginine and histidine. In one embodiment, the PTD domain consists of from about 7 to 10 amino acids, wherein at least 4 amino acids are lysine or arginine. In another aspect, the PTD domain consists of a sequence of about 7 to 10 amino acids and contains the sequence KX₁RX₂X₁, wherein X₁ is R or K and X₂ is any amino acid (SEQ ID NO:7). Such a peptide can be synthesized using techniques known in the art and chemically linked to a heterologous sequence. Alternatively, an oligonucleotide encoding SEQ ID NO:7 can be operably linked to a coding sequence for a heterologous polypeptide. The PTD domain described herein can be operably linked to a heterologous domain to generate a chimeric fusion molecule.

As used herein “chimeric fusion molecule” comprises a PTD domain (e.g., SEQ ID NO:1, 4, 6, or 7), if desired a signal sequence and a polypeptide of interest genetically fused together. The PTD domain and/or the signal sequence can be located 5′ or 3′ to the domain comprising/encoding the polypeptide of interest molecule.

The PTD domain consisting of SEQ ID NO:1, 4, 6, or 7 directs the transport of a peptide, protein, or molecule associated with the PTD from the outside of a cell into the cytoplasm of the cell. This can occur through macropinocytosis. Furthermore, a peptide that contains a PTD of the invention and additional amino acid sequences could be used to deliver molecules to cells (i.e., to deliver cargo) for the purposes of the present invention. The PTD of the invention (e.g., SEQ ID NO:1, 4, 6, or 7) may comprise of D- or L-amino acids.

The fatal neurodegenerative disorder variant-Creutzfeldt-Jakob disease (vCJD) in humans occurs following consumption of PrP^{Sc} contaminated tissue that converts cellular PrP^{C} into the disease causing, protease resistant isoform, PrP^{SC}. Importantly, several recent outbreaks of vCJD have occurred worldwide following consumption of BSE infected beef. Similar to the HIV-1 TAT protein transduction domain, prion proteins enter cells by stimulating lipid raft mediated, macropinocytosis. PrP^{C} contains an N-terminal cationic prion transduction domain that is both necessary and sufficient to stimulate macropinocytosis and induce cytoplasmic escape. Also identified herein is a PrP domain (residues 30-90) that, though unnecessary for infection of cells, enhances endosomal escape. The parallels between the TAT protein transduction domain and PrP are striking. Consistent with an underlying basis for host infection by consumption of PrP^{Sc} contaminated material, oral administration of TAT-β-galactosidase reporter protein results in penetration across the gut epithelium followed by redistribution into body tissues, including low levels in the brain. These observations are also consistent with reports that polyanionic compounds, such as heparin and PEI, can prevent PrP^{C} infection of cells and suggest that prophylactic administration of heparin-like compounds or macropinocytosis inhibitors during consumption of potentially contaminated material may aid with blocking oral uptake.

Protein transduction domains (PTDs) are short basic peptide sequences present in many cellular and viral proteins that mediate translocation across cellular membranes. The mechanism responsible for PTD-mediated membrane translocation varies among the various PTDs reported in the literature. For example, uptake by the retroviral TAT (transactivator of transcription) protein PTD requires cell surface-expressed glycosaminoglycans.

The HIV-1 TAT PTD has the unusual property to translocate into the cytoplasm of cells and has now been used to deliver a wide variety of protein and peptide cargo into cells both in vitro and in vivo. The TAT PTD transduction activity is dependent on an arginine and lysine rich domain (RKKRRQRRR-SEQ ID NO:1) that is required for cell surface binding to proteoglycans, stimulation of macropinocytosis and endosomal escape into the cytoplasm. TAT PTD transduction is dependent on electrostatic interactions with cell surface proteoglycans resulting in macropinocytotic uptake, a specialized form of fluid-phase endocytosis, followed by cytoplasmic release. The initial step in TAT PTD transduction requires an ionic interaction with cell surface glycosaminoglycans (GAGs).

Macropinosomes avoid fusion to lysosomes and are thought to traffic throughout the cell. Although cytoplasmic PrP is known to cause neurotoxicity, fusion of PrP^{Sc} containing macropinosomes to vesicles or the Golgi could result in a mixing environment conducive to conversion of endogenous, membrane-bound PrP^{C} to the pathological form, followed by trafficking to the cell surface. Because of its strict requirement for actin, the most commonly used inhibitors of macropinocytosis are the cytochalasins, particularly cytochalasin D. Macropinocytosis is also highly dependent on the activity of phosphatidylinositol (PI) 3-kinase (PI3K) and the activity of Rho family small GTPases, which regulate actin rearrangements. Inhibitors of PI kinases, such as wortmannin and LY294002, and Rho GTPases, such as toxin B, along with amiloride, an inhibitor of Na⁺/H⁺ exchange, can be used to inhibit macropinocytosis in cells.

Several promising molecular approaches in targeting the process of macropinocytosis have emerged recently, which target ARF- and Rho-family GTPases. Overexpression of ARF6 locked in its GTP-bound form, dominant-negative forms of the Rho family GTPases and the autoinhibitory domain of the Rac-dependent kinase PAK1, all result in an inhibition of macropinocytosis.

The invention demonstrates that the N-terminus of a prion protein contains a cationic PTD that interacts with the cell surface, inducing macropinocytosis and promotes escape from the macropinosome vesicle into the cytoplasm. The invention provides, based in part upon this information, methods of inhibiting prion infection by treating cells or subjects with an anionic agent the neutralizes the cationic charge of PrP and/or treating subjects with an inhibitor of macropinocytosis.

Similar to the TAT protein transduction domain, the invention demonstrates, in one embodiment, that coincubation of cells with anionic glycosaminoglycans prevents prion protein internalization and intracellular accumulation. In addition, specific inhibitors of macropinocytosis (e.g., 2-nitro-4-carboxyphenyl N,N-diphenylcarbamate (NCDC), hexodecylphosphocholine (HPC), U73122, phosphoinositide (PI) 3-kinase inhibitor-wortmannin, LY294002, and cytochalasin D, an inhibitor of actin polymerization) can function more efficaciously to prevent both the initial cellular uptake of PrP^{Sc} and reduce or eliminate cell-to-cell spreading.

The invention provides for the first time, that PrP enters cells under a mechanism similar to the mechanism of HIV-1 TAT protein transduction. Prion proteins contain an amino-terminal cationic transduction domain that stimulates lipid raft-dependent macropinocytosis and cytoplasmic escape. Moreover, PrP contains a second amino-terminal domain that enhances transduction into cells.

The invention demonstrates that treatment of cells being exposed to prion proteins with an anionic agent (e.g., heparin, an anionic polymer, and the like), neutralizes the cationic charge in the prion's PTD domain and prevents entrance or infection of cells. Accordingly, in one aspect, the invention provides that concomitant oral treatment with an anionic agent during consumption of infected meat can neutralize the prion's PTD and prevents its escape from the intestinal track into the blood stream and eventual entry into the CNS. In another example, treatment of infected subjects with anionic heparin, and/or with other anionic polymers, will neutralize and prevent further spread of the disease. Although the disclosure describes heparin in many specific examples as a model system, other anionic agents and polymers will work in a similar fashion. For example, other anionic agents include, but are not limited to, alkylaryl sulphonate, capryl imidazoline, dioctylester sodium sulphosuccinic acid, sodium lauryl sulphate, potassium lauryl sulphate, sodium alkylated aryl polyether sulphate, chondroitin sulfate B, heparin and other polysulphated polyanions (e.g., heparin sulfate and dermatan sulfate). Those of skill in the art will recognize that various “heparin” agents exist. For example, heparins as used herein include heparinoids, heparin derivatives and the like. Several heparin-like anionic molecules and heparin derivatives have been developed. For example, the capsular K5 polysaccharide from E. coli has the same structure as the heparin precursor N-acetyl heparosan. Chemical sulfation in N- and/or O-positions can generate heparin/HS-like molecules that can used in the methods and compositions of the invention.

In another aspect of the invention, macropinocytosis inhibitors can be used to prevent or treat prion infections. As described in further detail below, prion infections occur through a macropinocytosis uptake and processing by cells. Accordingly, inhibition of macropinocytosis will prevent uptake of prions. A number of macropinocytosis inhibitors are known in the art. For example, macropinocytosis inhibitors include, but are not limited to, phosphoinositide (PI) 3-kinase inhibitors (e.g., wortmannin), LY294002 and cytochalasin D. PI 3-kinase inhibitors have been shown to prevent complete formation of macropinosomes.

The methods and compositions of the invention are useful to treat or prevent infections by prions, prion variants, prion fragments, prion fusions, and analogues thereof having identity to SEQ ID NO:3 or a fragment thereof. Such variant, fragments, fusion, and analogues will have interactions or activities that are substantially the same as those of a full length or signal domain cleaved prion protein sequence and includes all forms of secondary structure. The term also includes prion surrogates, that is to say proteins which are not themselves prions but which have similar structure or exhibit similar behavior to prions. The term “PrP^{Sc} prion protein” is intended to have a similarly broad meaning but is limited to prion proteins which by virtue of their secondary or tertiary structure are enzyme resistant and includes conformations which are similarly enzyme resistant. Thus, the disclosure encompasses prion proteins and variant that are at least 80% identical, 85% identical, 90% identical, 95% identical, and 98% identical to SEQ ID NO:3.

In scanning the prP^{C} amino acid sequence for potential entry domains, a conserved N-terminal basic amino acid domain (residues 23-29) was identified and present in the mature, signal sequence cleaved, PrP^{C} protein (residues 23-231), that was similar to the PTD from HIV-1 TAT protein (FIG. 1a).

The invention also provides methods and compositions useful to prevent or inhibit prion-associated diseases and disorders. Prion-associated diseases, and disorders include all forms of spongiform encephalopathies. Characteristics of the spongiform encephalopathies include the appearance of the brain with large vacuoles in the cortex and cerebellum. Specific examples of prion-associated diseases and disorders include, but are not limited to, Scrapie in sheep, TME (transmissible mink encephalopathy) in mink, CWD (chronic wasting disease) in muledeer and elk, BSE (bovine spongiform encephalopathy) in bovines and particularly cows, CJD (Creutzfeld-Jacob Disease) in humans, GSS (Gerstmann-Straussler-Scheinker syndrome) in humans, FFI (Fatal familial Insomnia) in humans, Kuru in humans, and Alpers Syndrome in humans.

The fatal neurodegenerative disorder variant-Creutzfeldt-Jakob (vCJD) disease occurs following host exposure of PrP^{Sc} contaminated tissue resulting in conversion of cellular PrP^{C} (alpha helical) form into the disease causing, partially protease resistant PrP^{Sc} (β-sheet rich conformer). Fundamental mechanistic questions of how exogenous PrP^{Sc} protein infects cells and where conversion of cellular PrP^{C} to the pathological PrP^{Sc} form takes place have remained unclear. The invention demonstrates that similar to HIV-1 TAT, PrP^{C} contains a strong N-terminal transduction domain that was sufficient to direct cellular uptake and cytoplasmic release of a PrP-CRE recombinase fusion protein. Consistent with TAT, association of recombinant rPrP^{C} with the cell surface could be competed with soluble GAGs and endocytic uptake occurred by lipid raft-dependent macropinocytosis.

Moreover, inhibition of macropinocytosis still allowed PrP cell surface association, but prevented PrP^{Sc}-mediated conversion of PrP^{C} to the PrP^{Sc} form. PrP27-30 (residues 90-231), that lacks the N-terminal transduction domain, is still able to cause pathogenic conversion of PrP^{C}, suggesting that the second lysine-rich transduction motif between amino acids 100-109, is sufficient in the absence of the N-terminal domain to direct cellular entry. Regardless of one or two basic domains, inhibition of macropinocytosis blocks all forms of PrP^{Sc} present in RML brain homogenates from converting PrP^{C} to the PrP^{Sc} isoform.

Agents useful to inhibit prion infection in the methods of the invention can be identified using in vitro and in vivo techniques. For example, a test agent can be contacted with a cell culture before, concomitantly with, or after contact with an infectious prion (e.g., PrP^{SC}). The uptake of the infectious prion by the cells can then be measured using labelling techniques or by measuring the amount proteinase digestible PrP before and after contact with the test agent and a PrP^{Sc}. For example, where an agent is an effective inhibitor of prion protein uptake (i.e., having anti-prion activity) by cells, there will be a reduced amount of prion protein within the cell compared to a control (e.g., cells not contacted with the test agent). In one aspect, the infectious prion (e.g., PrP^{Sc}) is labelled with an agent that facilitates its detection. Various labels are known in the art.

The term “anti-prion activity” as used herein means an agent that inhibits or prevents the growth or proliferation of an infectious prion. For example, anti-prion activity includes the inhibition of uptake of infectious prions, prion release from macropinosomes, and/or the spread of infectious prions from one cell to another in in vitro cultures or in vivo from cell to cell or organ to organ.

The disclosure provides a method for inhibiting the uptake, infectivity and/or propagation of infectious prion by contacting the prion, a cell, or an organism with an inhibiting effective amount of an anionic agent and/or an inhibitor of macropinocytosis. The term “contacting” refers to exposing the prion, cell, or organism to an anionic agent and/or macropinocytosis inhibitor such that the anionic agent and/or inhibitor reduces or eliminates prion uptake by a cell or release of a prion in a macropinosome within a cell. An organism as used herein includes mammalian organisms such as primates, humans, bovines, porcines, equines and the like, Contacting of an organism with an anionic agent or macropinocytosis inhibitor of the disclosure can occur in vitro, for example, by adding the agent or inhibitor to a cell culture to test for susceptibility of the cell to an infectious prion in the presence and absence of an agent. Alternatively, contacting can occur in vivo, for example by administering the anionic agent and/or macropinocytosis inhibitor to a subject afflicted with a prion infection or susceptible to a prion infection. In vivo contacting includes both parenteral as well as topical. “Inhibiting” or “inhibiting effective amount” refers to the amount of anionic agent and/or macropinocytosis inhibitor that is sufficient to cause, for example, inhibition of prion uptake. The method for inhibiting the uptake and/or spread of prions can also include contacting the prion with the agent or inhibitor.

An anionic agent and/or macropinocytosis inhibitor of the disclosure can be administered to any host, including a human or non-human animal, in an amount effective to inhibit uptake and/or spread of prions. Thus, the anionic agents and macropinocytosis inhibitors are useful as anti-prion agents.

Any of a variety of art-known methods can be used to administer an anionic agent of the disclosure and/or a macropinocytosis inhibitor to a subject. For example, the agent or inhibitor of the disclosure can be administered parenterally by injection or by gradual infusion over time. The agent and/or inhibitor can be administered intravenously, intraperitoneally, intramuscularly, subcutaneously, intracavity, by inhalation, or transdermally.

In another aspect, an anionic agent and/or macropinocytosis agent can be formulated for topical administration (e.g., as a lotion, cream, spray, gel, or ointment). Such topical formulations are useful in treating or inhibiting prion presence or infections on the eye, skin, and mucous membranes such as mouth, vagina and the like. Examples of formulations in the market place include topical lotions, creams, soaps, wipes, and the like. The agents and inhibitors may be formulated into liposomes to reduce toxicity or increase bioavailability. Other methods for delivery of the anionic agents and macropinocytosis inhibitors include oral methods that entail encapsulation of the agent and/or inhibitor in microspheres or proteinoids, aerosol delivery (e.g., to the lungs), or transdermal delivery (e.g., by iontophoresis or transdermal electroporation). Other methods of administration will be known to those skilled in the art.

Preparations for parenteral administration of an agent or inhibitor of the disclosure include sterile aqueous or non-aqueous solutions, suspensions, and emulsions. Examples of non-aqueous solvents are propylene glycol, polyethylene glycol, vegetable oils (e.g., olive oil), and injectable organic esters such as ethyl oleate. Examples of aqueous carriers include water, saline, and buffered media, alcoholic/aqueous solutions, and emulsions or suspensions. Examples of parenteral vehicles include sodium chloride solution, Ringer's dextrose, dextrose and sodium chloride, lactated Ringer's, and fixed oils. Intravenous vehicles include fluid and nutrient replenishers, electrolyte replenishers (such as those based on Ringer's dextrose), and the like. Preservatives and other additives such as, other antimicrobial, anti-oxidants, inert gases and the like also can be included. Preparations for parenteral administration of an anionic agent and/or macropinocytosis inhibitor the disclosure include sterile aqueous or non-aqueous solutions, suspensions, and emulsions. Examples of non-aqueous solvents are propylene glycol, polyethylene glycol, vegetable oils (e.g., olive oil), and injectable organic esters (e.g. ethyl oleate). Examples of aqueous carriers include water, saline, buffered media, alcoholic/aqueous solutions, and emulsions or suspensions. Examples of parenteral vehicles include sodium chloride solution, Ringer's dextrose, dextrose, sodium chloride, lactated Ringer's, and fixed oils. Intravenous vehicles include fluid and nutrient/electrolyte replenishers (such as those based on Ringer's dextrose), and the like. Liquid and dry powder aerosols are also envisioned. Preservatives and additives such as, other antimicrobial agents, antioxidants, chelating agents, inert gases etc. can be included.

The disclosure provides a method for inhibiting prion uptake, release from macropinosomes, and/or spread by contacting or administering a therapeutically effective amount of an agent or inhibitor of the disclosure to a subject who has, or is at risk of having a prion infection. The term “inhibiting” means preventing or ameliorating a sign or symptoms of a disorder (e.g., subacute dementia, PrP immunoreactive plaques). Examples of subjects who can be treated in the disclosure include those at risk for, or those suffering from, a prion infection.

The term “therapeutically effective amount” as used herein for treatment of a subject afflicted with a prion infection or disorder associated with the infection means an amount of an anionic agent and/or macropinocytosis inhibitor sufficient to ameliorate a sign or symptom of the disease or disorder. For example, a therapeutically effective amount can be measured as the amount sufficient to decrease the amount of infectious prion in the central nervous system, in the blood stream or in a tissue sample. Typically, the subject is treated with an amount of the anionic agent and/or macropinocytosis inhibitor sufficient to reduce the amount of prion taken up by a tissue or propagated in a tissue or a symptom of a disorder associated with the infection by at least 50%, 90% or 100%. Generally, the optimal dosage of the agent or inhibitor will depend upon the type of infection, the location of the prion and factors such as the weight of the subject, sex, and degree of symptoms. Heparin, for example, is a well studied therapeutic agent. Dosages of heparin can be determined based upon the various toxicity and side-effects associated with the molecule (as well as derivative thereof). Nonetheless, suitable dosages can readily be determined by one skilled in the art. Typically, a suitable dosage is 0.5 to 40 mg/kg body weight, e.g., 1 to 8 mg/kg body weight.

If desired, a suitable therapy regime can combine administration of an agent or inhibitor of the disclosure with one or more additional therapeutic agents (e.g., a combination of various anionic agents, a combination of an anionic agent and a macropinocytosis inhibitor, a combination of macropinocytosis inhibitors and the like). The anion agent and/or inhibitor, other therapeutic agents, and/or antibiotic(s) can be administered, simultaneously, but may also be administered sequentially.

In another embodiment, an anionic agent and/or a macropinocytosis inhibitor can be used as preservatives or sterillants of animal beef or other tissue that may be subject to contamination. For example, the agent of inhibitor can be used as preservatives in processed foods.

The disclosure also provides a method for inhibiting the spread or infection of a prion by contacting the prion, a food product or a surface upon which a prion may be present with an inhibiting effective amount of an anionic agent and/or macropinocytosis inhibitor of the disclosure. The anionic agents and macropinocytosis inhibitors, kits, and preparations of the disclosure can be used therapeutically and prophylactically for biodefense against bioattacks and for use in areas of prion contamination of livestock. For example, the disclosure provides kits containing formulations comprising an anionic agent and/or a macropinocytosis inhibitor of the disclosure. The kits can be provided, for example, to a population living in an area of prion infected livestock.

The working examples are provided to illustrate, not limit, the invention. Various parameters of the scientific methods employed in these examples are described in detail below and provide guidance for practicing the invention in general.

### EXAMPLES

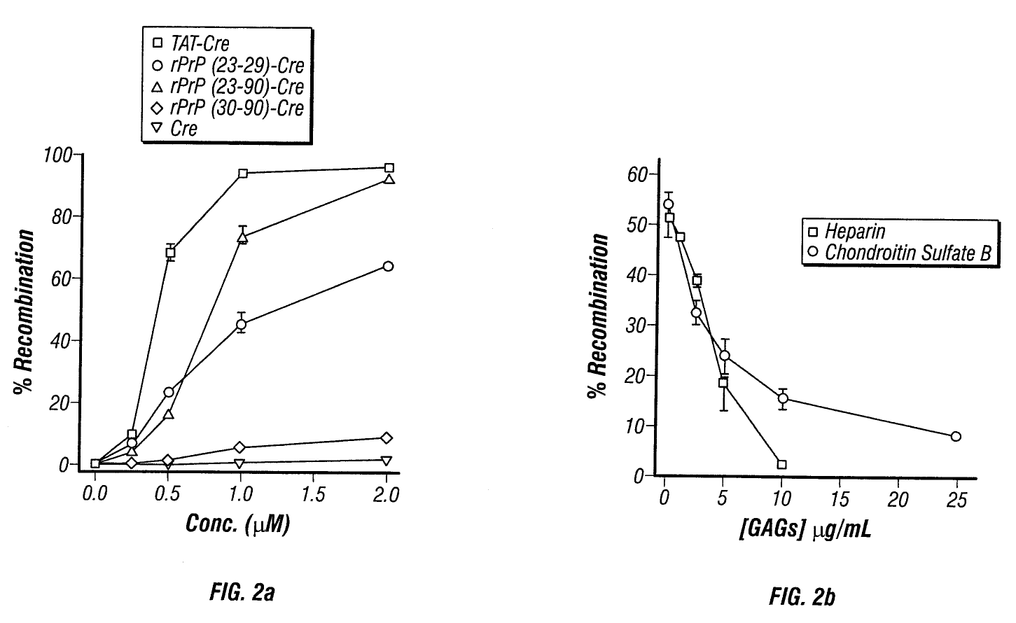

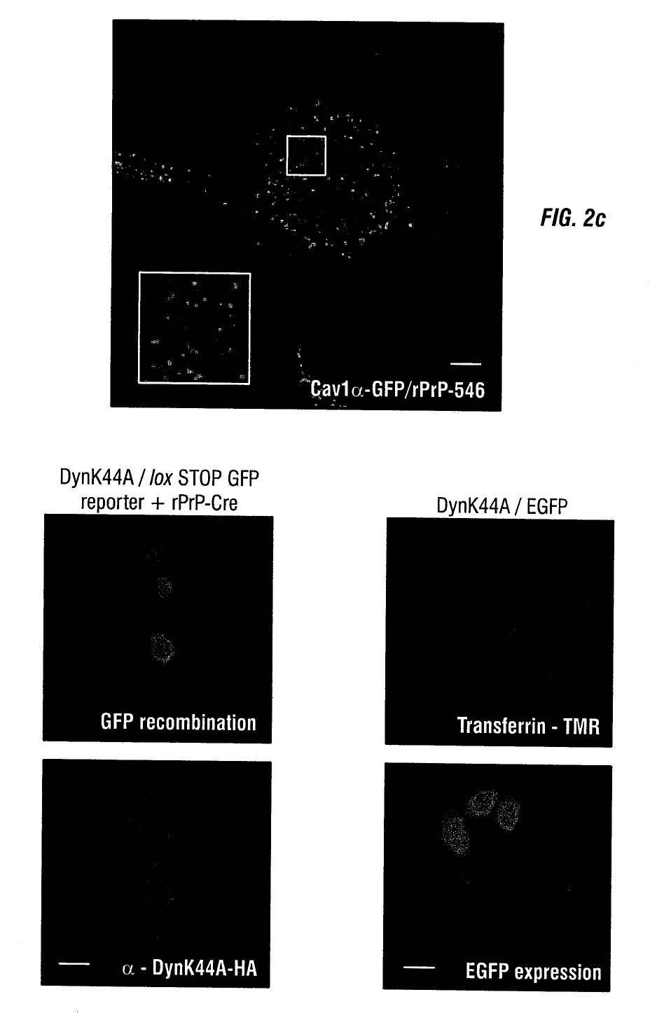

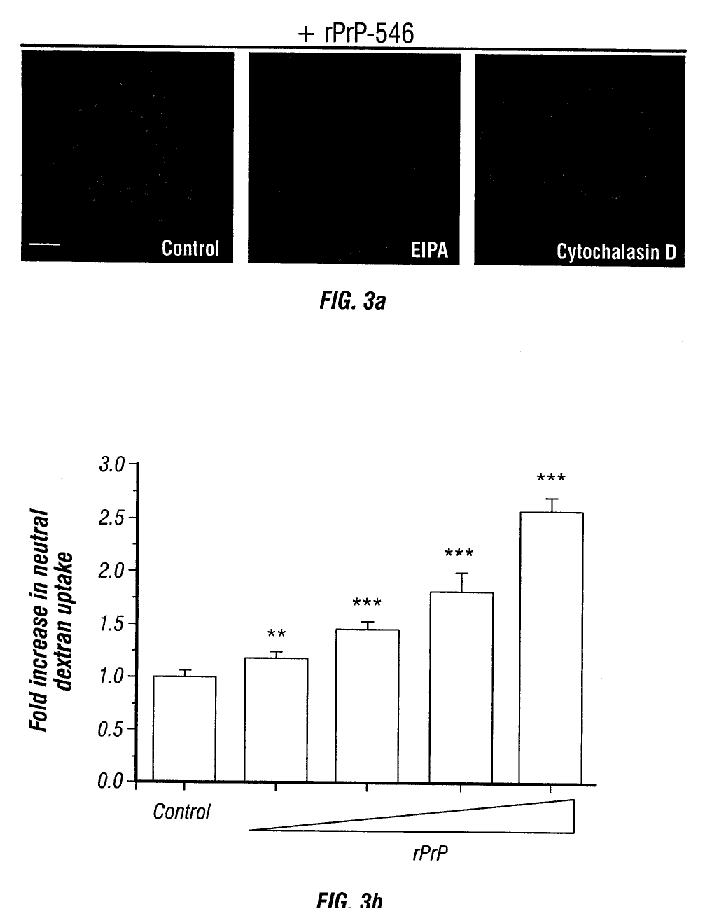

To ascertain the amino acid sequence requirements within the amino terminus for PrP entrance into cells, recombinant fusion proteins were generated with different PrP domains and Cre DNA recombinase as a reporter for cellular internalization (FIG. 1a). Exogenous recombinant TAT-Cre fusion protein can transduce into reporter cells and excise a transcriptional termination DNA segment from a loxP-stop-loxP GFP reporter gene to allow GFP expression, accordingly a similar strategy was used for the determination of PrP transduction domain. Consistent with TATCre, treatment of reporter cells with rPrP^{C}(23-90)-Cre or the putative N-terminal PrP transduction domain alone rPrP^{C}(23-29)-Cre resulted in the concentration-dependent recombination and expression of GFP (FIG. 2a). In contrast, both rPrP^{C}(30-90)-Cre and control Cre protein treatment failed to induce recombination above background levels. Moreover, co-incubation of cells with rPrP^{C}(23-90)-Cre protein with soluble GAGs, heparin or chondroitin sulfate B, inhibited protein uptake into cells and subsequent DNA recombination in a dose-dependent manner (FIG. 2b). These observations confirm the presence of a transduction domain at the N-terminus of PrP (residues 23-29) that is sufficient for both endocytic uptake and subsequent cytoplasmic escape of exogenously applied rPrP^{C}. Interestingly, the entire N-terminal domain of PrP (23-90), was more effective than just the basic PrP (23-29) domain at entering cells, suggesting the presence of additional elements that may enhance endosomal escape.

Endocytic uptake of PrPC has previously been reported to occur through either clathrin-dependent or caveolar-dependent forms of endocytosis. Since cellular uptake of the TAT PTD does not occur through either mechanism, we treated N2a cells expressing GFP tagged caveolin-1-α, a marker of caveolae, with fluorescent rPrP^{C}-546. Parallel to TAT PTD uptake, co-localization between rPrP^{C}-546 and caveolae was not detected by confocal imaging of live cells (FIG. 2c). Multiple forms of endocytosis, including clathrin- and caveolar-mediated endocytosis, require dynamin GTPase activity for vesicle formation at the cell surface and expression of dominant-negative dynamin-1 (Dyn^{K44A}) effectively blocks these endocytic pathways. Expression of Dyn^{K44A} in N2a cells expressing a loxP-stop-loxP GFP reporter plasmid failed to block cellular uptake of rPrP^{C}(23-90)-Cre and GFP expression (FIG. 2d). In contrast, Dyn^{K44A} expression inhibited uptake of a clathrin-mediated endocytosis marker, fluorescent-labeled transferring (FIG. 2e). These observations demonstrate that rPrP^{C} internalization occurs through a lipid raft-dependent process that is independent of both caveolar- and clathrin-mediated endocytosis.

TAT-fusion proteins and peptides enter cells by lipid raft-dependent macropinocytosis, a specialized actin-dependent, fluid phase endocytic process. To examine the potential involvement of macropinocytosis in rPrP^{C} uptake, N2a cells were treated with common macropinocytosis inhibitors EIPA, an analogue of amiloride which inhibits a Na+/H+ exchange specific for macropinocytosis, or cytochalasin D, an F-actin elongation inhibitor, prior to incubation with fluorescent-labeled rPrP^{C}-546 and confocal imaging of live cells. Treatment of N2a cells with both inhibitors prevented the cellular uptake of rPrP^{C}-546, but did not block its cell surface association (FIG. 3a). Interestingly, cell surface binding of TAT-fusion proteins stimulates macropinocytosis. To determine whether rPrPC cell surface binding can induce macropinocytosis, N2a cells were incubated with a fluorescent fluid phase macropinocytosis marker, 70-kDa neutral dextran-FITC, and increasing concentrations of rPrP^{C} (FIG. 3b). Neutral dextran was primarily taken up in N2a cells by amiloride sensitive macropinocytosis. Treatment of cells with rPrP^{C} protein induced a significant (p<0.02-0.002), concentration-dependent increase in dextran-FITC fluid-phase uptake over steady-state control levels (FIG. 3b). Taken together, these observations exclude both clathrin and caveolar endocytosis, and demonstrate that exogenous rPrP^{C} protein stimulates its own uptake by lipid raft-mediated macropinocytosis.

Cellular PrP^{C} and pathological PrP^{Sc} proteins have a markedly different C-terminal structural conformation with the latter having extensive β-sheet content. In experiments using recombinant PrP^{C} it remained unclear if pathologic PrP^{Sc} would behave similarly. To determine if PrP^{Sc} infects cells by macropinocytosis and that macropinocytosis contributed to the conversion of endogenous PrP^{C} to the PrP^{Sc} form, susceptible N2aPK125 cells were treated with RML PrP^{Sc}-infected murine brain homogenates and increasing concentrations of the macropinocytosis inhibitor EIPA.

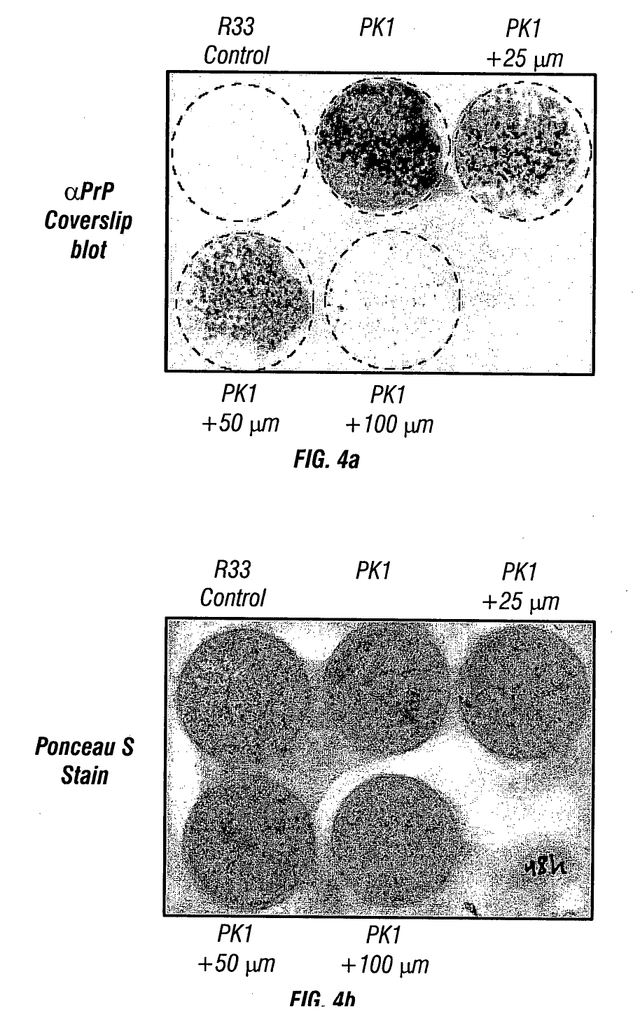

Treatment of N2aPK1 cells with RML PrP^{Sc}-infected brain homogenates for 48 h resulted in conversion of cellular PrP^{C} into the proteinase K resistant PrP^{Sc} form, whereas control non-susceptible N2aR33 cells were resistant to conversion (FIG. 4). Co-treatment of N2aPK1 cells with RML PrP^{Sc}-infected brain homogenate and EIPA for 48 h resulted in an EIPA concentration-dependent inhibition of PrP^{C} to PrP^{Sc} conversion in vivo (FIG. 4). Similar results were obtained for 72 h co-treatment of N2aPK1 cells with RML PrP^{Sc}-infected brain homogenates. These observations in this cell culture system demonstrate that conversion of cellular PrP^{C} to the pathologic Prp^{SC} requires macropinocytosis.

Small amounts of cytoplasmic PrP have been reported to cause neurotoxicity. Consistent with this notion, a small proportion of internalized rPrP was detected escaping from macropinosomes. In parallel to TAT-fusion proteins/peptides that distribute throughout tissues in rodent models, including low levels in the brain, the molecular and cell biology results presented here demonstrate a similar mechanism underlying the basis for host exposure to PrP^{Sc} contaminated material. Broad based polyanionic compounds, such as heparin and PEI are effective to inhibit PrP^{Sc} infection of cells by sequestration of the N-terminal basic domain identified here. In addition, inhibitors of macropinocytosis can function to prevent initial cellular uptake of PrP^{Sc}. The invention demonstrates a molecular mechanism for pathological PrP^{Sc} protein infection of cells by macropinocytosis and conversion of cellular PrP^{C} protein.

Recombinant Proteins. rPrP^{C} was expressed in BL21 pLysS cells (Novagen) from a pET28 vector (Novagen) by IPTG induction for 3 h. Cells were resuspended in cold buffer W (50 mM Tris pH 8.0, 250 mM NaCl, 5 mM EDTA, 1 mM PMSF, 10 μg/mL leupeptin, 0.1 mM aprotinin, 10 μg/mL DNase 1, 10 μg/mL lysozyme), sonicated and inclusion bodies collected by centrifugation at 30,000×g, 20 min and solubilized in buffer G (6 M GdmCl, 20 mM Tris pH 8.0, 50 mM Na₂HPO₄, 100 mM NaCl, 10 mM reduced glutathione, 10 mM imidazole). Cleared lysates were incubated overnight at RT with shaking, purified on Ni-NTA column, washed with a gradient of buffer G and buffer B (10 mM Tris pH 8.0, 100 mM Na₂HPO₄, 0.1 mM oxidized glutathione, 10 mM imidazole) at ratios of 6:0, 5:1, 4:2, 3:3, 4:2, 5:1 and 0:6, respectively. rPrP^{C} was eluted in 20 mM Tris pH 8.0, 1 M imidazole and an on-column oxidation was repeated twice. Fractions were buffer exchanged into 50 mM Hepes pH 7.0, 100 mM NaCl and 5% glycerol and concentrated by ultrafiltration. rPrP^{C}(23-90)-Cre, rPrP^{C}(30-90)-Cre, rPrP^{C}(23-29)-Cre, TAT-Cre, and control Cre were purified. rPrP^{C} and TAT-Cre proteins were conjugated with Alexa546 or Alexa488 (Molecular Probes) at a 1:1 molar ratio.

Recombination Assays. Reporter T cells containing an integrated loxP-STOP-loxP GFP expression gene were treated with recombinant protein in the presence/absence of heparin (Sigma) or chondroitin sulfate B (Sigma) RPMI for 1 h at 37° C., 5% CO₂. Cells were trypsinized, washed 2× in PBS and replated in RPMI+10% FBS for 18 h followed by FACS for GFP positive cells. Cell death was measured by propidium iodide staining, and flow cytometry analysis.

Confocal microscopy. Murine N2a neuroblastoma cells were grown on glass coverslips and exposed to 2.0 μM rPrP^{C}-Alexa546 for 2 h, washed and live cell images were acquired at a depth through the middle of the nucleus using a BioRad MRC1024 confocal microscope. To determine colocalization with caveolae, N2a cells were transiently transfected with 0.2 μg caveolin-1-GFP expression vector using Fugene-6, washed and incubated with rPrP^{C}-Alexa546 for 2 h. N2a cells were pretreated with either 50 μg/mL heparin (Sigma), 5 mM nystatin (Sigma) 100 μM cytochalasin D (Sigma) or 100 μM EIPA (Sigma) for 30 min, prior to adding 2.0 μM rPrP^{C}-Alexa546 for 2 h. For co-localization studies, N2a cells were treated with 2.0 μM rPrP^{C}-Alexa546 and 2.0 μM TAT-Cre-Alexa488. After 2 h, cells were washed and corresponding fluorescent confocal images for rPrP^{C}-Alexa546 fluorescence (PMT1) and TAT-Cre-Alexa488 fluorescence (PMT2) were obtained.

Dynamin-1 (K44A). N2a cells were transfected at a ratio of 10:1 with DynaminK44A-HA (pDyn^{K44A}-H) expression plasmid and pZ/EG loxP-STOP-loxP GFP expression vector. After 24 h, cells were treated with 2.0 μM rPrP^{C}(23-90)-Cre for 1 h, trypsinized, washed, replated in DMEM+10% FBS for 18 h and analyzed for GFP by FACS. Immunohistochemistry using anti-HA antibody (Babco) followed by anti-mouse TRITC secondary antibody (Jackson Labs) was used to verify Dyn^{K44A} expression. Control Dyn^{K44A} and pEGFP vector (Stratagene) (10:1) expressing cells were incubated in serum-free media for 4 h prior to addition of 25 μg/mL transferrin conjugated tetramethylrhodamine (Molecular Probes) for 15 min.

Quantification of Macropinocytosis. N2a cells were incubated in DMEM+ (serum free DMEM, 0.1% BSA and 10 mM Hepes pH 7.4) at 4° C. for 30 min. To measure macropinocytosis, 0.5 mg/mL 70 kDa neutral dextran-FITC (Molecular Probes) was added to cells treated with increasing concentrations of recombinant rPrP^{C} protein (0, 0.25, 0.5 1.0, or 2.0 μM), incubated for 30 min and analyzed by FACS. Background dextran fluorescence uptake was inhibited by incubation at 4° C. for 30 min. Fold increase in dextran uptake was calculated after subtracting background fluorescence from each sample.

Cell-based PrP^{Sc} infectivity assay. Infectivity assay were performed as follows: 1×10⁴ susceptible N2aPK1 cells and resistant N2aR33 cells, maintained in opti-MEM (Gibco) plus 10% FBS, were exposed to a 10-5 dilution of RML PrP^{Sc}-infected murine brain homogenates for 48 h in the presence or absence of 25, 50, 100 μM EIPA (Sigma). Cells were grown to confluence, washed and split 1:10. Replating at 1:10 was repeated twice. After the last passage 5×10⁴ cells were plated onto 25 mm Thermanox coverslips (Nunc, Fischer Scientific), grown for 4 d and then blotted onto a nitrocellulose membrane (Biorad, 0.45 μm pore size) soaked in lysis buffer (1% Triton X100, 0.5% deoxycholate, 150 mM NaCl, 50 mM Tris-HCl, pH 8.0). The membranes were dried for 1 h at 37° C., incubated with 0.5 μg/mL PK in lysis buffer for 90 min at 37° C. followed by treatment with 3M guanidinium thiocyanate, 10 mM Tris-HCl (pH 8.0) for 10 min. After washing, the membranes were blocked in 5% non-fat dry milk and probed with 6H4 anti-PrP antibody (1:1000, Prionics, Zurich) followed by HRP-conjugated anti-mouse IgG1 antibody. PrPSc positive colonies were visualized using Supersignal West Pico ECL reagent (Pierce Biotechnology).

A number of embodiments of the invention have been described. Nevertheless, it will be understood that various modifications may be made without departing from the spirit and scope of the invention. Accordingly, other embodiments are within the scope of the following claims.

1. A method of inhibiting infection by an infectious prion, comprising:
contacting a cell susceptible to infection with a prion with an inhibiting effective amount of an agent selected from the group consisting of an anionic agent, a glycosaminoglycan, an agent that sequesters cholesterol, a macropinocytosis inhibitor and any combination thereof, prior to, concomitant with, and/or following contact of the cell with the prion, for a sufficient time and under sufficient conditions such that the anionic agent inhibits uptake of the prion.

2. The method of claim 1, wherein the contacting is in vivo.

3. A method of inhibiting the infectivity of a prion comprising contacting a sample suspected of containing a prion with an agent selected from the group consisting of an anionic agent, a glycosaminoglycan, an agent that sequesters cholesterol, a macropinocytosis inhibitor and any combination thereof.

4. The method of claim 3, wherein the sample is a meat sample.

5. The method of claim 4, wherein the meat sample is a bovine meat sample.

6. The method of claim 5, wherein the sample is a surface of an object.

7. A method of treating a subject having or at risk of becoming infected with an infectious prion, comprising administering to the subject an agent selected from the group consisting of an anionic agent, a glycosaminoglycan, an agent that sequesters cholesterol, a macropinocytosis inhibitor and any combination thereof in an amount sufficient to inhibit prion infectivity or spread.

8. The method of claim 1, wherein the anionic agent is a heparin, a heparin derivative, and/or a heparinoid agent.

9. The method of claim 1, wherein the anionic agent neutralizes the cationic nature of the N-terminal portion of a prior protein.

10. The method of claim 1, wherein the anionic agent is selected from the group consisting of alkylaryl sulphonate, capryl imidazoline, dioctylester sodium sulphosuccinic acid, sodium lauryl sulphate, potassium lauryl sulphate, sodium alkylated aryl polyether sulphate, heparin and chondroitin sulfate B.

11. The method of claim 1, wherein the macropinocytosis inhibitor is a PI kinase inhibitor, a Rho GTPase inhibitor, and/or an inhibitor of Na+/H+ exchange.

12. The method of claim 1, wherein the macropinocytosis inhibitor is selected from the group consisting of EIPA, amiloride, and cytochalasin D.

13. The method of claim 1, wherein the macropinocytosis inhibitor is selected from the group consisting of wortmannin and LY294002.

14. The method of claim 1, wherein the method comprises a combination of an anionic agent and a macropinocytosis inhibitor.

15. A method of inhibiting the production of a pathological prion protein comprising
contacting a cell susceptible to infection with a prion or infected with a pathological prion with an inhibiting effective amount of an agent selected from the group consisting of an anionic agent, a glycosaminoglycan, an agent that sequesters cholesterol, a macropinocytosis inhibitor and any combination thereof, prior to, concomitant with, and/or following contact of the cell with the prion, for a sufficient time and under sufficient conditions such that the anionic agent inhibits uptake of the prion.

16. The method of claim 15, wherein the contacting is in vivo.

17. The method of claim 15, wherein the anionic agent is a heparin, a heparin derivative, and/or a heparinoid agent.

18. The method of claim 15, wherein the anionic agent neutralizes the cationic nature of the N-terminal portion of a prion protein.

19. The method of claim 15, wherein the anionic agent is selected from the group consisting of alkylaryl sulphonate, capryl imidazoline, dioctylester sodium sulphosuccinic acid, sodium lauryl sulphate, potassium lauryl sulphate, sodium alkylated aryl polyether sulphate, heparin and chondroitin sulfate B.

20. The method of claim 15, wherein the macropinocytosis inhibitor is a PI kinase inhibitor, a Rho GTPase inhibitor, and/or an inhibitor of Na+/H+ exchange.

21. The method of claim 15, wherein the macropinocytosis inhibitor is selected from the group consisting of EIPA, amiloride, and cytochalasin D.

22. The method of claim 15, wherein the macropinocytosis inhibitor is selected from the group consisting of wortmannin and LY294002.

23. The method of claim 15, wherein the method comprises a combination of an anionic agent and a macropinocytosis inhibitor.

24. A composition for use in inhibiting prion infectivity in a subject comprising an agent selected from the group consisting of an anionic agent, a glycosaminoglycan, an agent that sequesters cholesterol, a macropinocytosis inhibitor and any combination thereof in unit dose form.

25. The composition of claim 24, wherein the anionic agent is a heparin, a heparin derivative, and/or a heparinoid agent.

26. The composition of claim 24, wherein the anionic agent neutralizes the cationic nature of the N-terminal portion of a prior protein.

27. The composition of claim 24, wherein the anionic agent is selected from the group consisting of alkylaryl sulphonate, capryl imidazoline, dioctylester sodium sulphosuccinic acid, sodium lauryl sulphate, potassium lauryl sulphate, sodium alkylated aryl polyether sulphate, heparin and chondroitin sulfate B.

28. The composition of claim 24, wherein the macropinocytosis inhibitor is a PI kinase inhibitor, a Rho GTPase inhibitor, and/or an inhibitor of Na+/H+ exchange.

29. The composition of claim 24, wherein the macropinocytosis inhibitor is selected from the group consisting of EIPA, amiloride, and cytochalasin D.

30. The composition of claim 24, wherein the macropinocytosis inhibitor is selected from the group consisting of wortmannin and LY294002.

31. The composition of claim 24, wherein the method comprises a combination of an anionic agent and a macropinocytosis inhibitor.
